(19) United States
(12) Patent Application Publication
(10) Pub. No.: US 2013/0066367 A1
(43) Pub. Date: Mar. 14, 2013

(54) SUTURE BONE ANCHOR

(71) Applicant: Slobodan Tepic, Zurich (CH)

(72) Inventor: Slobodan Tepic, Zurich (CH)

(21) Appl. No.: 13/611,615

(22) Filed: Sep. 12, 2012

Related U.S. Application Data
(63) Continuation of application No. 12/107,068, filed on Apr. 21, 2008, now abandoned.
(60) Provisional application No. 60/913,274, filed on Apr. 21, 2007.

(51) Int. Cl. A61B 17/04

(52) U.S. Cl. 606/224; 606/232

(57) ABSTRACT

A bone anchor provides stable, durable anchorage of sutures used for repair of soft tissues torn, or cut, away from bones. The anchor includes a plurality of prongs angled in the direction of loading. The prongs-are inserted into holes drilled in the bone. The angled nature of the prongs causes them to dig into the bone under functional loading. A suture is attached to the anchor via an integral bead and led through the anchor eyelet. In use, the suture does not glide anywhere at its interface to the anchor, nor does if flex on itself at a knot. Instead, it only flexes around the pillar of the eyelet with a fully circular cross-section.

### CROSS-REFERENCES TO RELATED APPLICATIONS

This application claims priority to U.S. Provisional Patent Application Ser. No. 60/913,274, filed Apr. 21, 2007.

### BACKGROUND OF THE INVENTION

1. Field of the Invention

The invention relates to bone anchors commonly used in surgical repairs of soft tissue attachments to bones, such as torn ligaments, tendons or joint capsules.

2. Discussion of Related Art

Trauma and corrective orthopedic interventions frequently involve reattachment of soft tissues to bones. Such reattachments are complicated by the very different mechanical properties of the two types of tissue. While soft tissue to soft tissue repairs are done with needles and sutures, fractured bones are fixed with screws, screws and plates, pins or nails. When facing the problem of fixing a torn ligament or tendon lacking sufficient remnant on the bone, surgeons are in need of so-called bone anchors. Bone anchors are implants which are fixed to the bone by one or the other means typical of those used for bone repair. The bone anchor includes a hole or an eyelet to which a suture is tied. Anchors are either completely inserted into bone or partially left above it. In most cases the attachment is expected to heal, i.e. the function of the anchors is temporary. The suture materials can be either stable in the body or bioresorbable. Both options are available for the bone anchors as well.

The most typical shape of a state of the art bone anchor is a bone screw with a hole drilled through its head. With the distance of the anchor's point of suture attachment above the bone, the pull on it creates a bending moment, risking loosening of the anchor through either direct damage to the bone, or its gradual loss through bone resorption. The suture can also fail, usually at its point of attachment, by either fatigue due to bending or wear due to movement.

FIG. 1 shows a state of the art bone anchor 1 inserted into bone 2. The eyelet 3 of the bone anchor 1 is a few millimeters above the bone surface. Most of the applications for bone anchors are in bone near joints, where the thickness of the cortical bone 4 tends to be very small. The shaft of the anchor 5 is screwed into soft, weak cancellous bone 6. Pull 7 on the suture 8 tied in the eyelet 3 creates a bending moment 9 which can loosen the anchor 1 from the weak bone. The sweeping movement 10 of the suture when in use is either causing the suture to turn around the side 11 of the eyelet 3 or flexing at the knot 12. Either of these causes wear and ultimately failure.

FIG. 2 shows another state of the art anchor 13 inserted into cancellous bone 6 below the level of the cortical bone 4. Pull 7 with sweeping movement 10 on the suture 14 causes it to bend over the edge 15 of the anchor hole 16. Damage at that point is predictably high to both the bone and the suture.

### SUMMARY OF THE INVENTION

The present invention substantially overcomes the deficiencies of existing bone anchors by providing a bone anchor having at least two prongs positioned along the expected line of pull. The effect of the bending moment is minimized which prevents loosening of the anchor. According to one aspect of the invention, at least two holes are drilled into the bone approximately in the line of pull expected to be exerted on the anchor. According to another aspect of the invention, the holes are drilled precisely with the aid of a drill guide. According to another aspect of the invention, the holes are drilled at an acute angle to the bone surface causing the anchor to dig into the bone when exposed to functional loading. According to another aspect of the invention, an anchor having at least two prongs is inserted into bone by tapping its prongs into the precisely drilled holes. According to another aspect of the invention, the anchor is formed of a metal, preferably titanium.

According to another aspect of the invention the eyelet for suture attachment has well-defined conditions at the interface prevent unintended movement which could lead to wear and fatigue of the suture material. According to another aspect of the invention, the suture also satisfies certain conditions, particularly related to the diameter and arrangement of its fibers, for improved wear and performance.

### BRIEF DESCRIPTION OF THE DRAWINGS

FIG. 1 is a cross sectional view of a state of the art bone anchor with the eyelet above the bone.

FIG. 2 is a cross sectional view of a state of the art bone anchor fully inserted into the bone.

FIG. 3 is a perspective view of an anchor according to an embodiment of the invention.

FIG. 4 is a cross sectional view of the anchor of FIG. 3 taken at the level of the eyelet.

FIG. 5(a) is a cross sectional view of a prong of the anchor of FIG. 3 inserted in bone.

FIG. 5(b) is a cross sectional view of a drill guide according to an embodiment of the present invention.

FIG. 5(c) is a cross sectional view of an anchor according to an embodiment of the invention inserted in bone.

FIG. 6(a) illustrates wrapping and unwrapping of the suture over the pillar of the anchor.

FIG. 6(b) is a cross sectional view of the anchor of FIG. 3 with the suture inserted.

FIG. 7 is a cross sectional view of an anchor according to another embodiment of the present invention with a starting and finishing hole for the suture.

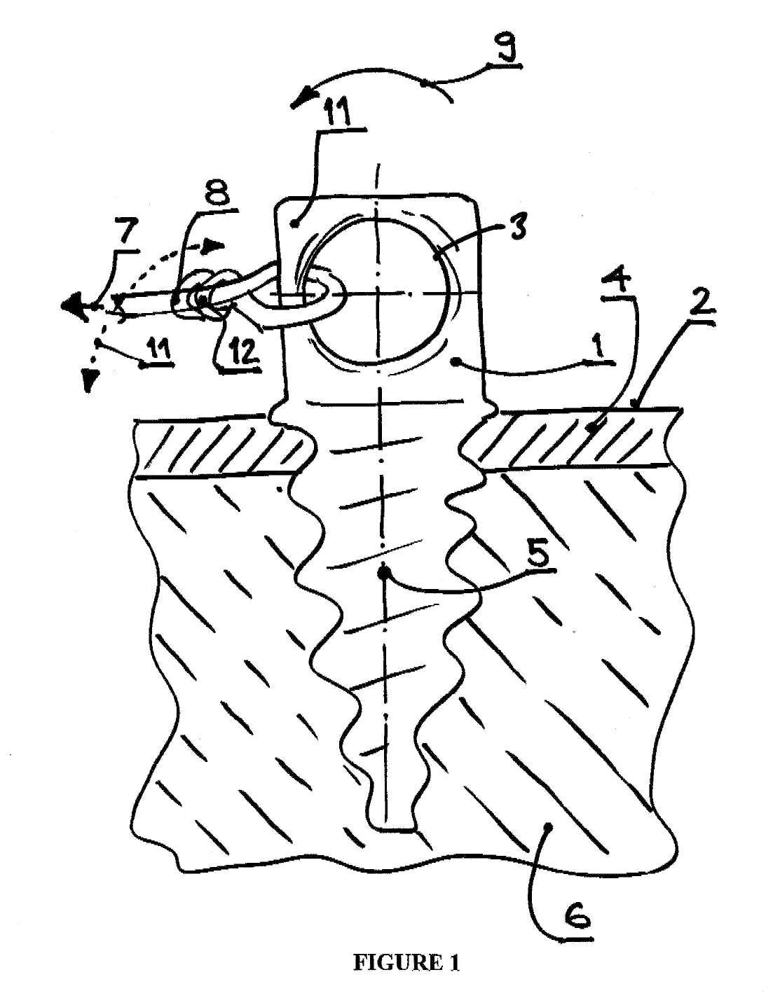

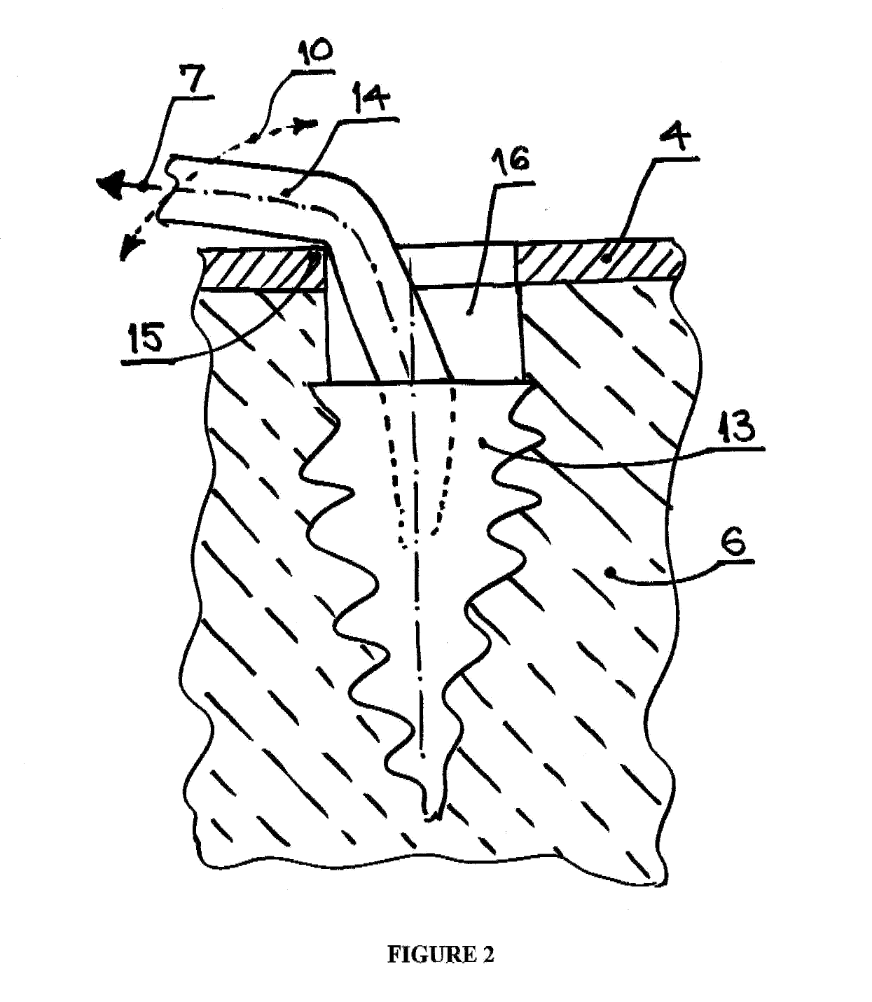

### DETAILED DESCRIPTION

The present invention provides a bone anchor having improved performance and reduced wear and failure of sutures. An embodiment of the invention includes a bone anchor having a plurality of aligned prongs. Any number of prongs may be used, but two or three are preferred. To install the anchor, a plurality of holes are drilled along the line of expected pull of the suture. A drill guide may be used to properly position the holes. The prongs of the anchor are inserted in the holes.

According to an embodiment of the invention, the bone anchor further includes a structure for attaching the suture which reduces wear and failure. The structure includes a first shaped hole with rounded edges through which the suture passes. The suture is able to flex on the edges. A second hole is provided for attaching the end of the suture. A third hole may also be used for attaching the second end of the suture.

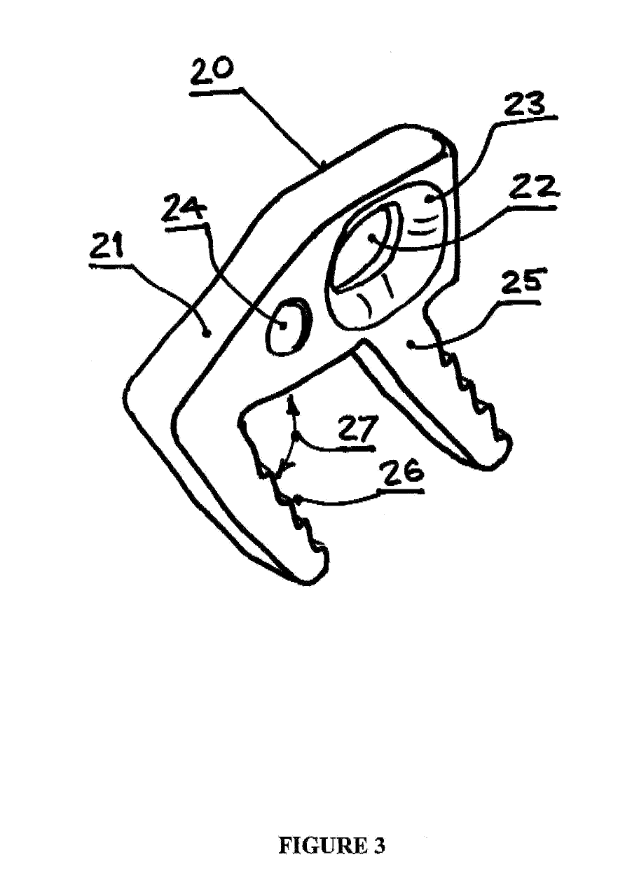

FIG. 3 shows a perspective view of an anchor 20 according to an embodiment of the invention. The bone anchor 20 is formed of a metal, preferably titanium. The faces are substantially planar.

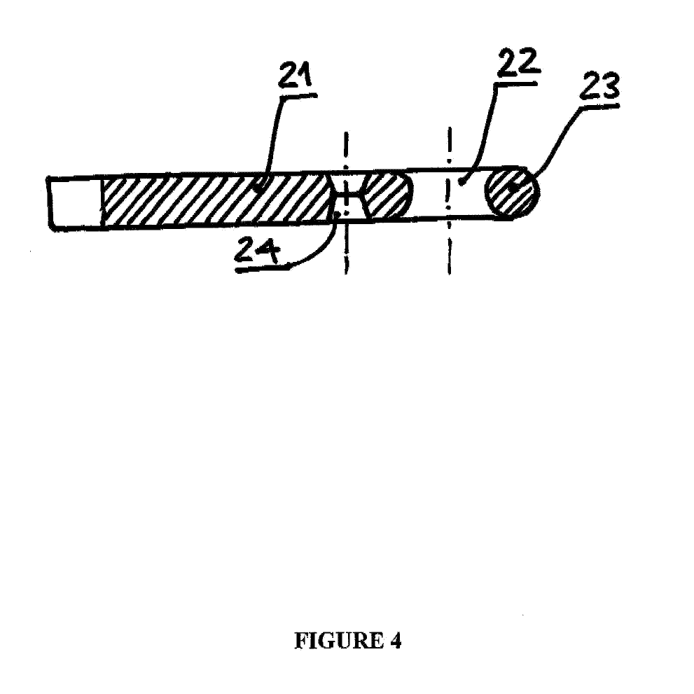

The part of the anchor intended to remain above the bone 21 is provided with an opening 22 having all of its edges well rounded. The pillar 23 closing the side of the opening 22 is also well rounded. On the opposite side of the opening 22 there is a hole 24 which is conical in shape from both faces, i.e. the diameter decreases from both faces of the anchor towards the middle. FIG. 4 shows a cross section of the anchor 20 taken through the upper part 21 at the middle of the opening 22 and the hole 24. The cross section of the pillar 23 is completely circular. The side of the hole 22 opposite to the pillar 23 is also well rounded.

The prongs 25 of the anchor are substantially square in cross section. They are provided with fine teeth 26 to improve the grip in the bone and prevent pullout. The prongs 25 are angled 27 in the direction of anticipated pull so that the anchor tends to dig into the bone. The angle 27 is preferably in the range from 50 to 70 degrees and, more preferably, about 60 degrees.

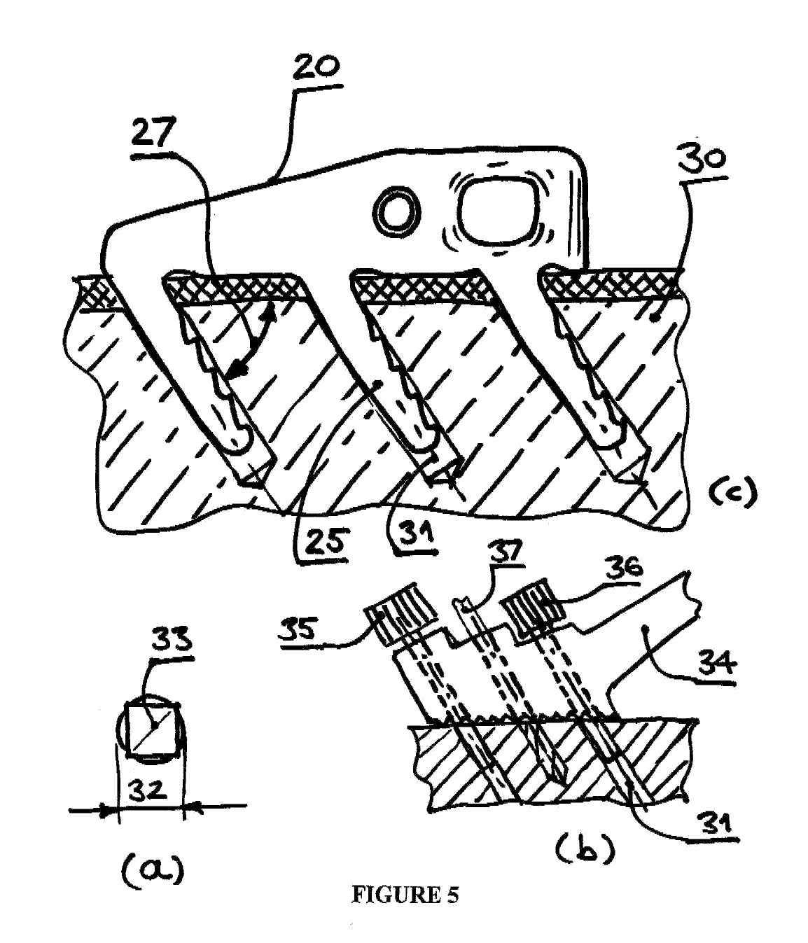

FIG. 5(c) shows the anchor 20 inserted in bone 30. The embodiment shown in FIG. 5 has three prongs, whereas the embodiment of FIG. 3 had only two. The prongs 25 of the anchor 20 are inserted into pre-drilled holes 31. As illustrated in FIG. 5(a), the diameter 32 of the holes 31 is slightly smaller—by 0.05 to 0.1 mm—than the diagonal 33 of the prongs 25. The anchor is tapped into place. The slightly smaller diameter of the holes allows for easy insertion and provides initial stability needed to prevent bone resorption around the prongs 25. Bone remodeling will fill the holes and result in fully integrated prongs.

The process for drilling the holes is illustrated in FIG. 5(b). A drill guide 34 is used to precisely position and angle the holes. Once the first hole is drilled, a positioning pin 35 is inserted through the guide 34 into the hole. The next hole is then drilled and another positioning pin 36 is inserted to define the position for drilling the last hole. Preferably, the middle hole is drilled last, but the holes could be drilled in any order.

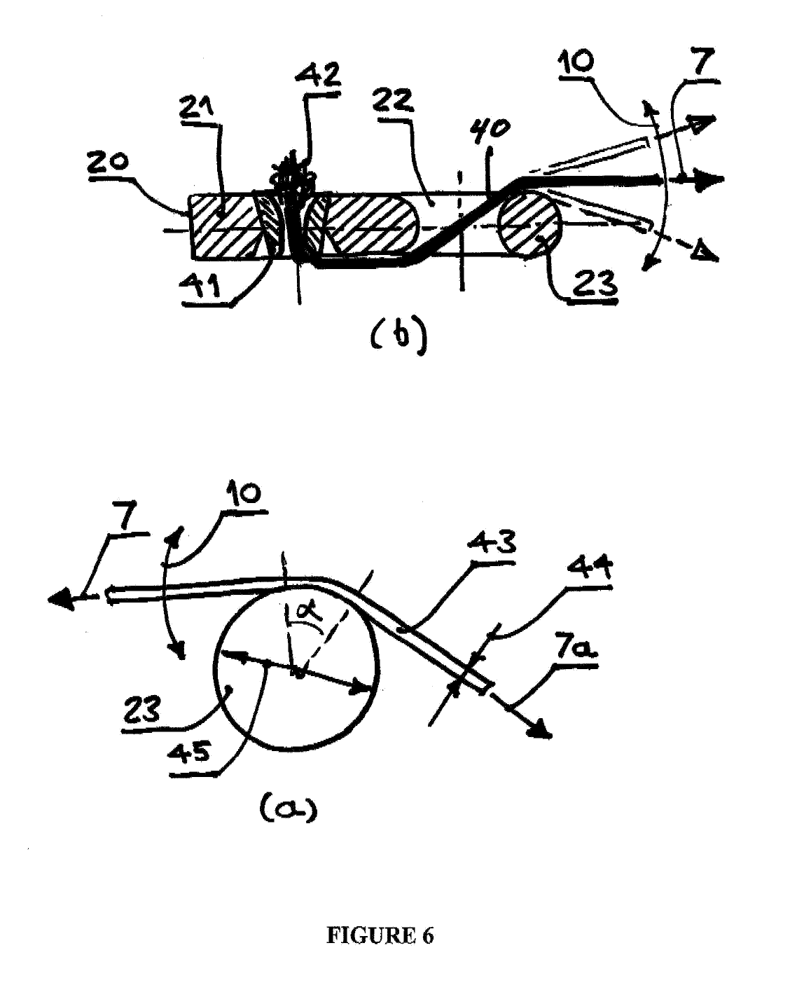

FIG. 6(b) shows a cross section of the anchor 20 with the suture 40 in place. The suture is preferably provided with a tubular bead 41 on an end opposite the needle. The bead 41 has a conical outer diameter which matches the conical inner diameter of the hole 24 of the anchor 20. The inner diameter of bead 41 is sized to accommodate the suture 40 and increases in diameter towards the ends. A knot 42 in the suture cooperates with the decreasing inner diameter of the bead to prevent pullout of the suture through the bead 41. The exit of the inner diameter bead is well rounded so as to prevent damage to the suture as it bends from the bead and along a face of the anchor. The suture 40 then passes through the opening 22 and bends over the pillar 23. When in use, as the pull 7 on the suture sweeps 10, the suture 40 simply bends around the pillar 23. Any gliding motion between the suture and the anchor comes solely from the elasticity of the suture. Gliding motion between the suture and the anchor is limited to portion of suture between the exit from the bead 41 and the bend on the pillar 23. Since strong sutures typically used in these applications are also very stiff, gliding motion is minimized. If the magnitude of the pulling force 7 is very high, the suture can be wrapped a full turn around the pillar 23. Thus, the magnitude of the force 7a (in FIG. 6(a)) on the face of the anchor can be significantly reduced.

The suture is preferably of a multifilament type without braiding or bonding of filaments. FIG. 6(a) represents bending of individual filaments of the suture 40 about the pillar 23. An individual filament 43 has a diameter d denoted by 44. The diameter D of the pillar 23 is denoted by 45. Maximum strain experienced by the filament bending over the pillar is approximately equal to d/D. If the fatigue limit on the strain in an individual fiber is εₘₐₓ, the relative diameters of the pillar 23 and filaments 43 can be determined. The diameter of the pillar should be:

D>d/εₘₐₓ.

With the best expectations of high performance polymeric fibers, the fatigue limit on the strain in the fiber is on the order of 0.015, thus the diameter of the pillar D should be at least 60 times larger than the diameter of the filament d. The strongest filaments of e.g. highly oriented polyethylene are on the order of 0.015 mm in diameter. For those the pillar should have at the least the diameter of about 1 mm. However, this would leave no capacity to resist any tension. A factor of 2 in the diameter, i.e. a pillar of 2 mm diameter would allow maximum tension in fatigue to be about one half of its nominal value—a reasonable compromise.

Alternatively, if the fatigue strain at expected number of cycles in use be εₘₐₓ, the fiber diameter d, and the factor for allowed functional tension k. Then the diameter of the pillar, 23, of the anchor should be D>k(d/εₘₐₓ). Expressed in terms of the radius of curvature, R, of the edges: R>(k/2)(d/εₘₐₓ). Conversely, if the diameter of the anchor pillar D is given, one can determine that the fiber diameter d should be: d<(D/εₘₐₓ)/k.

State of the art sutures are either monofilament or multifilament, braided in one or the other way. Neither type can offer satisfying performance at the suture anchor. For the monofilament fibers the radius of the pillar required is simply not possible in most situations, i.e. those sutures will predictably fail in use. Braiding as conventionally done will effectively increase the diameter of the fiber and will also lead to failure. Another serious drawback of braiding is the increased risk of infection—bacteria within a braided suture are not accessible to immune system cells and can thus remain a threat as long as the suture is in the tissue. Technical reasons for braiding are mostly related to the ease and reliability of the knots, which need to be tied to complete the repair. Pre-assembly of the suture with a bead 41 eliminates the need to tie an anchor knot at one end. Alternatively, all or a portion of the multifilament suture can be held together with a gelatin or other substance which will dissolve within the body. This makes the suture easier to use and to tie, yet allows the filaments to separate in order to achieve improved wear and failure resistance.

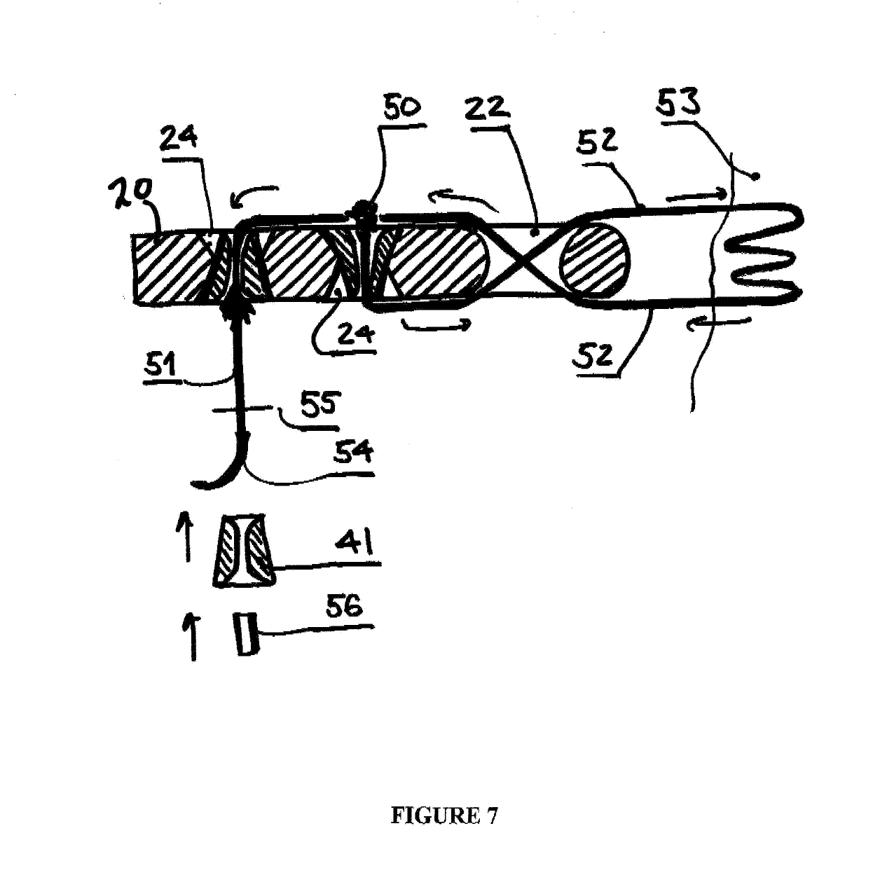

FIG. 7 shows another embodiment of the anchor 20 of the present invention. In this embodiment, the anchor 20 has two holes 24 one of which can be used for the start 50 and the other for the end 51 of the suture 52. Once the suture has been anchored in the tissue 53 it is passed through the opening 22 of the anchor and through the second one of the holes 24. The needle 54 is cut off 55 a second bead 41 is threaded over the suture, placed into its hole, and a blocking knot is tied and pushed to the exit end of the second bead. The knots can be secured by melting over it a polymer sleeve 56 of melting temperature lower than that of the suture itself. For example if the suture is made from oriented, high modulus, ultra high molecular weight polyethylene with melting temperature of about 150 deg C., the sleeve can be made from a low molecular weight polyethylene melting at about 110 deg C.

Preferred fiber for use with the anchor of the invention is that of oriented, high modulus, ultra high molecular weight polyethylene, such as DYNEEMA® from DSM, Netherlands, or SPECTRA® from Honeywell, USA. Preferred diameter of the fiber is between 10 and 20 micrometers, more preferably about 15 micrometers. Fibers are left free from each other, as in yarn; i.e. no diffusion bonding nor braiding is used in production. One end is fused, preferably with an aid of low molecular polyethylene, and supplied with a needle. The other end is supplied with a bead, a knot is tied behind it and secured/overmolded with low molecular polyethylene.

Other suitable polymeric fibers are polyethylene teraphthalate (polyester), polyamide (NYLON®), aramid (KEVLAR®), or silk. Resorbable fibers can also be used, e.g. those based on polylactic acid, polyglycolic acid or polydioxanone.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. A suture anchor comprising:
a base having an opening to accommodate a suture; and
at least two prongs for insertion into bone extending from the base.

2. The suture anchor of claim 1 wherein the base further includes at least one hole positioned away from the opening for fixation of a suture end.

3. The suture anchor of claim 1, wherein the prongs form an acute angle with the base.

4. The suture anchor of claim 3, wherein the prongs form an angle in the range of 50 to 70 degrees.

5. The suture anchor of claim 1, wherein each of the at least two prongs include a plurality of teeth.

6. The suture anchor of claim 1, wherein at least one edge of the base at the opening is rounded with a radii R equal or bigger than (k/2) (d/εₘₐₓ), wherein d is the diameter of individual filaments in the suture, εEₘₐₓ is the allowed strain in fatigue of the suture material, and k is the factor allowing for suture tension in cyclic use.

7. The suture anchor of claim 6, wherein the thickness of the anchor is equal to 2R and is in the range of 0.5 to 3.5 mm.

8. The suture anchor according to claim 2, further comprising a suture bead, sized to fit within the at least one hole, attachable to one end of a suture.

9. The suture anchor of claim 1, further comprising a suture attached to the anchor, wherein the suture includes a plurality of independent filaments.

10. The suture anchor of claim 9 wherein the diameter d of the independent filaments of the suture is smaller or equal than 2R εₘₐₓ/k, wherein R is a radius at the edges of the anchor opening, εₘₐₓ is the allowed strain in fatigue of the suture material, and k is the factor allowing for suture tension in cyclic use.

11. A suture for use with the bone anchor comprising:
at least one filament having a first end and second end;
a suture bead connected to the first end of the at least one filament; and
a needle connected to the second end of the filament, wherein the diameter of the needle is smaller than the diameter of the suture bead.

12. The suture of claim 11 wherein the at least one filament includes a plurality of independent filaments.
